(19) United States
(12) Patent Application Publication
(10) Pub. No.: US 2015/0342746 A9
(43) Pub. Date: Dec. 3, 2015

(54) SYSTEM, METHOD AND APPARATUS FOR SIMULATING INSERTIVE PROCEDURES OF THE SPINAL REGION

(71) Applicant: Milan Radojicic, Los Gatos, CA (US)

(72) Inventor: Milan Radojicic, Los Gatos, CA (US)

(73) Assignee: Neurosyntec Corp., Los Gatos, CA (US)

(21) Appl. No.: 13/726,403

(22) Filed: Dec. 24, 2012

Related U.S. Application Data
(63) Continuation-in-part of application No. 13/236,635, filed on Sep. 19, 2011, now abandoned.
(60) Provisional application No. 61/384,457, filed on Sep. 20, 2010.
(60) Provisional application No. 61/679,920, filed on Aug. 6, 2012.

(51) Int. Cl. A61F 2/44

(52) U.S. Cl. CPC A61F 2/442

(57) ABSTRACT

Sensor information is received that indicates insertion of an insertion mechanism into a mechanical body. A virtual representation of the spinal region is generated. The insertion mechanism is represented graphically, as part of the virtual representation, as the insertion mechanism is inserted into the mechanical body

### RELATED APPLICATIONS

This application is a continuation-in-part of U.S. patent application Ser. No. 13/236,635, filed on Sep. 19, 2011; which claims benefit of priority to Provisional Patent Application No. 61/384,457; all of the aforementioned priority applications being hereby incorporated by reference in their respective entirety.

This application also claims benefit of priority to Provisional Patent Application No. 61/679,920; the aforementioned priority application being hereby incorporated by reference in its entirety.

This invention was made with Government support under (Award No. 1214752) awarded by the National Science Foundation. The Government has certain rights in this invention.

### FIELD OF THE INVENTION

Disclosed embodiments relate to a system, method and apparatus for simulating insertive procedures of the spinal region.

### BACKGROUND

Insertive spinal procedures such as epidurals require the attending practitioner to be skilled. The margin of error in such procedures is often very small, and an inexperienced hand can cause significant injury. While the need exists for the practitioner to have skill, there exists little ability for the practitioner to acquire the skill and training, other than live patient trials.

### BRIEF DESCRIPTION OF DRAWINGS

FIG. 1A, a frontal view of a mechanical system for simulating a spinal region, according to an embodiment.

FIG. 1B illustrates a cross sectional view of FIG. 1A, along line A-A, according to an embodiment.

FIG. 1C illustrates an insertion mechanism for use in connection with a mechanical body such as shown and described with FIG. 1A and FIG. 1B, according to an embodiment.

FIG. 1D illustrates an embodiment in which a mechanical system of FIG. 1A though FIG. 1C is used in connection with a virtual simulation of a spinal region, according to an embodiment.

FIG. 2 illustrates an example system for virtually representing a simulation of a spinal region puncture in connection with a physical model such as described with FIG. 1A through FIG. 1D.

FIG. 3 illustrates a method for generating a virtual representation of a spinal region puncture based on a mechanical simulation, under an embodiment.

FIG. 4 illustrates a method a method for simulating the presence of an anatomical hazard when generating a virtual representation of a spinal region puncture based on a mechanical simulation, under an embodiment.

FIG. 5 is a block diagram that illustrates a computer system upon which embodiments described herein may be implemented.

### DESCRIPTION

Numerous embodiments described herein relate generally to enabling virtual and/or physical simulation of insertive procedures of the spinal region.

As used herein, the term “spinal region” (or variants such as “spine region”) refers to the spine (including the pelvic, sacral, lumbar, thoracic, cervical or craniocervical regions), as well as surrounding skin and tissue.

Some embodiments include a mechanical body, one or more sensors, and one or more processors that are coupled to the mechanical body. The mechanical body can include a spinal element and a synthetic tissue layer. The one or more processors that communicate with the one or more sensors to detect insertion of an insertion device into the mechanical body. The one or more processors operate to provide a virtual representation of a spinal region that corresponds to the mechanical body. The virtual representation can represent the insertion device as it is inserted into the mechanical body. The virtual representation can be based on a movement and/or position of the insertion mechanism in relation to the spinal element and the synthetic tissue layer of the mechanical body.

In another embodiment, sensor information is received that indicates insertion of an insertion mechanism into a mechanical body. A virtual representation of the spinal region is generated. The insertion mechanism is represented graphically, as part of the virtual representation, as the insertion mechanism is inserted into the mechanical body.

Still further, some embodiments include a mechanical body that includes a spinal element, one or more synthetic tissue layers, a plurality of sensors and a communication link. The sensors may be structured to detect insertion of an insertion mechanism. The communication links may be configured to communicate an output of the plurality of sensors to a computing system in real-time.

Still further, some embodiments described herein provide a simulation environment that can mimic normal and abnormal cerebrospinal anatomy and physiology. Among other benefits, examples described herein allow for training of medical personnel (e.g., student doctors, nurses etc.) in the use of neurological insertion devices (e.g., subdural and epidural needles, catheters, endoscopes), such as common in use of anesthesiology and pain management. In conventional practice, the training of such medical personnel requires actual human patients, and mistakes in the application of such insertion devices can be devastating to the patient and costly to the provider.

Examples described herein can provide a simulation environment for training medical personnel in the use of insertion devices about the spinal regions of the cerebrospinal system. Some examples described herein mimic the effect of cardiac, respiratory and body movements on the generation of the cerebrospinal fluid pressure wave and flow.

Other examples described herein can provide a simulation environment for a cranial access system. Still further, examples described herein can be used as a training device for spinal region punctures, intrathecal catheter placement, infusion experiments and other surgical interventions.

Additionally, examples can simulate the presence of anatomic constraints and pathological masses and scarring, to allow the user to develop skill. Examples described herein can be designed to teach skills to prevent common complications, such as over-drainage leading to spinal headache, as well as traumatic and dry taps, herniation syndromes, and injury to the conus medullaris, nerve roots and/or blood vessels.

One or more embodiments described herein provide that methods, techniques and actions performed by a computing device are performed programmatically, or as a computer-implemented method. Programmatically means through the use of code, or computer-executable instructions. A programmatically performed step may or may not be automatic.

One or more embodiments described herein may be implemented using programmatic modules or components. A programmatic module or component may include a program, a subroutine, a portion of a program, or a software component or a hardware component capable of performing one or more stated tasks or functions. As used herein, a module or component can exist on a hardware component independently of other modules or components. Alternatively, a module or component can be a shared element or process of other modules, programs or machines.

Furthermore, one or more embodiments described herein may be implemented through the use of instructions that are executable by one or more processors. These instructions may be carried on a computer-readable medium. Machines shown or described with figures below provide examples of processing resources and computer-readable mediums on which instructions for implementing embodiments of the invention can be carried and/or executed. In particular, the numerous machines shown with embodiments of the invention include processor(s) and various forms of memory for holding data and instructions. Examples of computer-readable mediums include permanent memory storage devices, such as hard drives on personal computers or servers. Other examples of computer storage mediums include portable storage units, such as CD or DVD units, flash or solid state memory (such as carried on many cell phones and consumer electronic devices) and magnetic memory. Computers, terminals, network enabled devices (e.g., mobile devices such as cell phones) are all examples of machines and devices that utilize processors, memory, and instructions stored on computer-readable mediums. Additionally, embodiments may be implemented in the form of computer-programs, or a computer usable carrier medium capable of carrying such a program.

Mechanical System

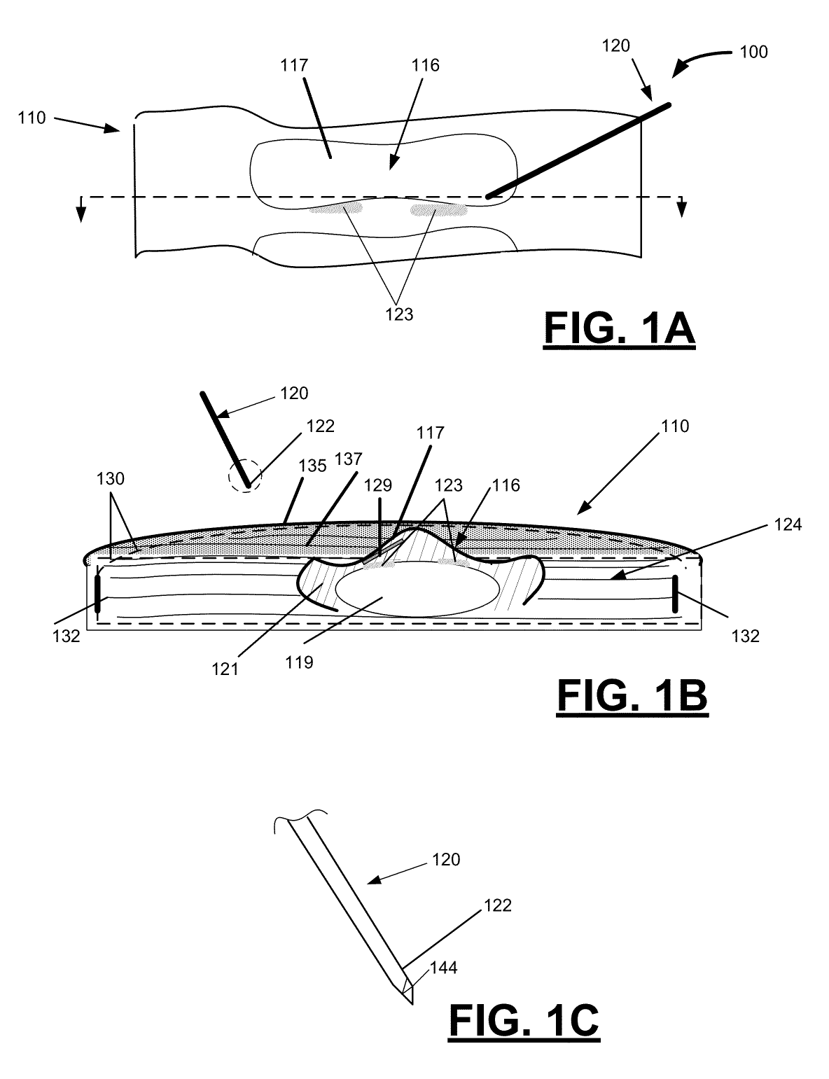

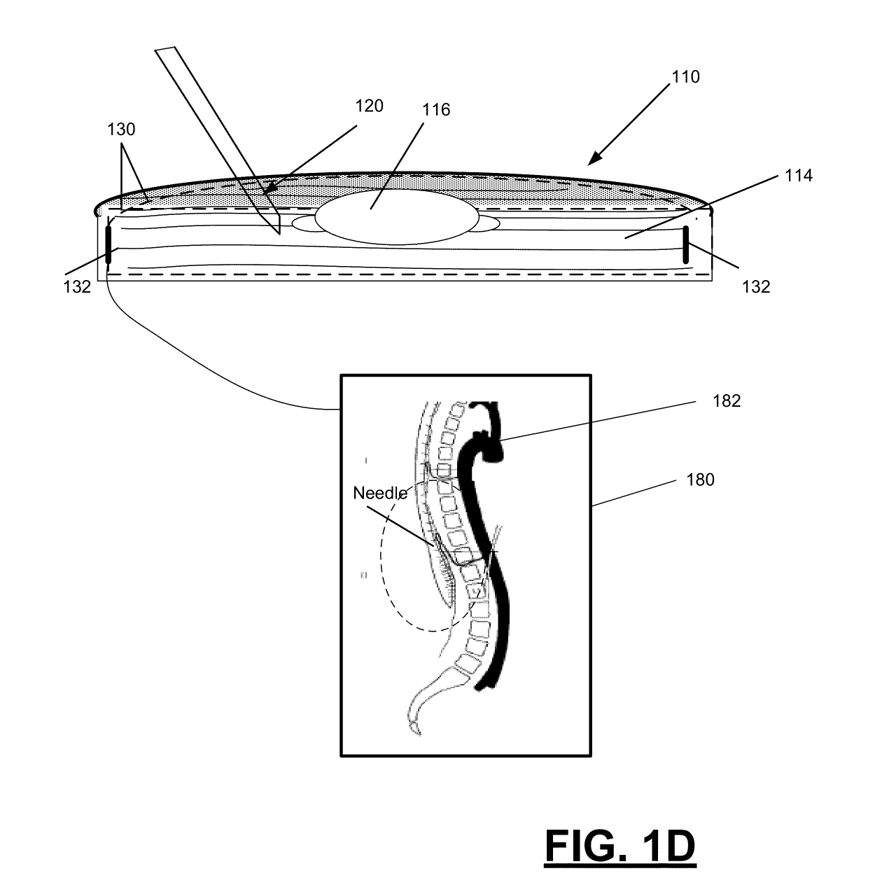

FIG. 1A through FIG. 1C illustrate example mechanical system for simulating a spinal region, according to various embodiments. In FIG. 1A, a frontal view of the mechanical system 100 is shown. FIG. 1B illustrates a cross sectional view of FIG. 1A, along line A-A. FIG. 1C illustrates an insertion mechanism for use in connection with a mechanical body such as shown and described with FIG. 1A and FIG. 1B, under an embodiment. Among other uses, the mechanical system 100 as shown by FIG. 1A through FIG. 1C can be used to train the physical ability of practitioners (e.g., doctors) to apply spinal regions injections. As further described with FIG. 2 and elsewhere, some embodiments further provide for the mechanical system 100 top optionally be used with a virtual computing environment such as described with an example of FIG. 2.

With reference to FIG. 1A and FIG. 1B, the mechanical system 100 can include a mechanical body 110 and an insertion mechanism 120. In one implementation, the insertion mechanism 120 is specialized for use with mechanical body 110 and/or other simulation environments. In variations, the insertion mechanism 120 can be mufti-functional, so as to not otherwise be specifically designed for the mechanical body 110. For example, the insertion mechanism 120 can be a model or replication of a Touhy type needle such as used in medical procedures of the spine. Alternatively, the insertion mechanism 120 can bean endoscope or catheter.

In an embodiment, the mechanical body 110 includes a spinal column or physical model 116 that is affixed within the body and surrounded by synthetic tissue. The body 110 can be used in, for example, a vertical or erect upright position or in a recumbent position so as to simulate an actual use environment as well as the effects of gravity on the user. The size of the mechanical body 110 can be selected to range from adult male, adult female, adolescent, child etc. In variations, the sensors 111 can be placed along the hard and/or soft tissue, such that deformations in a physical coordinate system can mapped to a corresponding virtual coordinate system.

With specific reference to FIG. 1B, the mechanical body 110 can include elements for simulating a human spinal region. In an example of FIG. 1B, the mechanical body 110 includes the spinal model 116 and one or more tissue layers 124. The one or more tissue layers 124 can also include an exterior skin layer 125. The spinal model 116 can be designed in the shape and dimension of a human spine or portion thereof. The tissue layers 124 can also be structured to provide tactile feedback in response to the penetration by the insertion mechanism 120. In some embodiments, the tactile feedback can simulate, through the insertion mechanism 120, the tactile feel of human tissue in response to insertion of a needle, plunger or catheter. Accordingly, the tissue layer 124 can include density, elasticity (or rigidity) and/or other physical properties that replicate human tissue. As an addition or alternative, the exterior layer 125 can include surface tension of human skin. The depth of the tissue layer 124 in relation to the spinal model 116 can also be designed to simulate human form. Moreover, the tissue layer 124 can have variable physical properties to simulate the different thicknesses of human tissue between the skin and the spine. For example, the density and elasticity of the tissue layers 124 can change in accordance with human tissue of the spinal region (e.g., to reflect ligaments etc.).

The tissue layers 124 can be formed from materials such as latex, plastic or rubber. The mechanical body 110 can also be modularized, so that portions of the mechanical body 110 can be replaced or combined with other portions. For example, portion of the tissue layer 124 can be replaceable with new material, as the mechanical body is worn down with use.

As shown further by FIG. 1B, the tissue layer 124 of the mechanical body 110 includes (i) the exterior layer 135, which is a synthetic representation of skin, (ii) the interior layers 137, which can include a synthetic representation of the subcutaneous tissue, and/or one or more layers of synthetic ligament tissue. The synthetic ligament tissue can include, for example, synthetic representations of one or more of supraspinous ligament, interspinous ligament, and ligamentum flavum. Other thicknesses of the mechanical body that can be punctured with the insertion mechanism include epidural space containing the internal vertebral venous plexus, dura, arachnoid, and the subarachnoid space. In one implementation, the physical model of the body 110 provides for the insertion mechanism 120 to simulate a puncture needle that pierces, in order, some or all of the following layers: skin, subcutaneous tissue, supraspinous ligament, interspinous ligament, ligamentum flavum, epidural space containing the internal vertebral venous plexus, dura, arachnoid, and finally the subarachnoid space. Accordingly, the interior layer 137 can be structured to simulate subcutaneous tissue and other interior layers.

In some embodiments, the tissue layer 124 is formed from layers of synthetic elastomeric material. As mentioned, the layers can be varied in physical properties to mimic actual tissue. Thus, variations in the synthetic material can be used to simulate the real-life tactile feel of human tissue at the spinal region. For example, the synthetic material can be toughened when used to form the synthetic ligament tissue so that the insertion mechanism 120 “pops” when entering the ligamentum flavum.

As an addition or alternative, the mechanical body 110 can be physically modeled into a partial human form. For example, the exterior layer can be contoured to reflect human shape and form, as well as landmarks such as buttocks. For example, the mechanical body 110 can be provided as a whole body mannequin, or a partial mannequin that represents a regional section of a human.

In more detail, the spinal model 116 includes shell 117, and tube 119 representing the spinal cord. In one implementation, the shell 117 is implemented as an s-shaped beam that is bendable. Rubber discs can be spaced between the vertebrae sections of the shell 117. In variations, additional anatomic structure such as facet joints, which are a target for pain management procedures can also be included (e.g., scar tissue, tumors, spinal injuries or abnormalities etc.) The tube 119 may be formed from elastomeric material. To model adults along the spinal cord between L1 vertebrae and pelvis, for example, the tube 119 may be approximately 2 cm in width, but the width of the spinal cord can vary in humans depending on the location. Thus, the width of the tube 119 can be varied depending on the region that is being mimicked, and variations in the width can be made to further mimic other regions of the spinal cord along the length of the spine.

As an addition or variation, the physical model 110 can be implemented to include anatomical abnormalities. For example, the body 110 can include a tube representing a large vessel such as the aorta. The placement of such a bendable tube, in addition to sensors in the tissue layer 124, enable simulations in which the operator has the ability to detect deformation in the soft tissue which may or may not occur with no deformation of the hard tissue. For example, the operator may interact with the body 110 by pressing hard and deforming the tissue layer 124. This act would simulate a doctor pushing on a patient's abdomen to deform the intestines, without affecting the vertebrae which would remain stationary.

In an embodiment, the tube 119 is placed within a medium 121 that simulates a dural sac. The medium 121 can be formed from, for example, rigid plastic, glass or elastomeric material along a substantial portion of the shell 117.

A penetrable region 123 to the dural sac can be modeled with the inclusion of a flexible membrane, such as formed by elastomeric material that can be resealed to allow for multiple punctures. As shown by FIG. 1A, the penetrable region 123 can be placed adjacent the shell 117, corresponding to the real-world location where a needle can be inserted. The penetrable material can be designed to be consumable and replaceable.

It will be appreciated that the complexity and degree of physical modeling for the mechanical body 110 can vary, depending on design selections. For example, the medium 121 and/or penetratable region 123 can be omitted in some implementations. Moreover, some implementations can add additional simulative elements of the human body, such as arterial flow within the body. In such an embodiment, an additional elastomeric tube (not shown) can be affixed to the tube 119 (spinal cord) in lengthwise fashion to provide arterial blood flow simulation. An additional or alternative tube (not shown) can be placed in the tissue layer 124 to represent, for example, a large artery such as the aorta. In one implementation, fluid can be pumped through the additional tube to simulate pulsatile flow. For example, a wave form generating pump (not shown) can be connected to the additional arterial tube. In implementation, the additional tube can be approximately less than 1.5 mm to simulate the actual size of a spinal artery. A larger tube (not shown) can be used to simulate an aorta. In a simplest form, one implementation provides for a single tube that runs lengthwise along the ventral surface of the tube 119 (spinal cord). In more realistic mechanical simulation, a network of bifurcating tubes can be used to simulate the tortuous arterial anatomy of the spinal cord. Still further, an intermediate version that is preferable would include a single ventral tube, simulating the anterior spinal artery, along with two dorsal tubes, simulating the posterior spinal arteries. The modeled spinal arteries would have inner diameters of about 0.1 to 1.5 mm. Pulsatile flow allows use of the system as a phantom for visualization probes, such as ultrasound or related modalities, which otherwise would necessitate in vivo models.

While an example described with FIG. 1A to FIG. 1C provides for mechanical simulation of a variety of human structures, some examples provide for some features encountered by insertion mechanism 120 to be virtually simulated. For example, large and fine neurovascular structures can be provided for in a virtual environment. Likewise, human fluid flows, such as aortic flows or cerebrospinal fluid flow can also be virtualized.

In an embodiment, the pulsitic pump can include a profile that is based on a digitized arterial waveform to confirm a realistic pulsatile waveform. A cam profile with a single or multiple waveform profile could be constructed. Alternatively, a pegs on a spindle could be positioned together and offset to simulate the peaks of the arterial dicrotic waveform. In another variation, a waveform generating pump can be provided.

As an addition or alternative, a venous system could be modeled with an inflatable compartment or balloon within the spinal cord elastomeric model, the inflation of which can be manually or computer controlled. As the venous congestion increases, the inner balloon or inflatable compartment would be expanded.

As another alternative or addition, the cerebrospinal fluid production is modeled with a influx of fluid into a compartment within the medium 121 (synthetic dura), but outside of the tube 119 (spinal cord) and vascular assembly.

As another alternative or addition, subdural scarring can be simulated with a mesh material 129 provided between the tube 119 (spinal cord) and the medium 121 (dura).

In variations, multiple fluid circuits or regions can be physically simulated in the mechanical body 110. By way of example, these include artery or veinous flows, lymph fluids, and/or cerebrospinal fluid, as well as cysts or cavities. As an addition or alternative, some of these fluids and other real-world aspects can be replicated virtually in a virtual environment.

Insertion Mechanism

The insertion mechanism 120 can correspond to any elongated and pointed member that can insert into or puncture the synthetic tissue layers of the mechanical body 110. For example, the insertion mechanism 120 can be provided as a needle or plunger. The insertion mechanism 120 can be structured to simulate devices such as a Touhy needle, a lumbar catheter, or a needle and catheter device. The insertion mechanism 120 can also physically simulate other surgical tools, such as catheters, steerable needles or endoscopes can be utilized.

With reference to FIG. 1C, some embodiments provide for the insertion mechanism 120 to include a tip 122 and, optionally, one or more sensor elements 144. The tip 122 can be sufficiently sharp and rigid to pierce synthetic material such as the tissue layer 124 and/or the penetrable regions 123 of the mechanical body 110.

In an example of FIG. 1C, the sensor elements 144 are positioned at or near the tip 122. In variations, the sensor element can be positioned along the body, or proximal to the tip (but apart from the tip). The sensor elements 144 can be part of the sensor system 140, as described below. As further described, the sensor elements 144 can include one or more of (i) a sensor set that detects elements or thicknesses of the body 110 and/or forms elements of a sensing system which includes sensors on the mechanical body 110, (ii) sensor trigger to trigger sensors of the mechanical body 110 so as to identify the position of the insertion mechanism 120 within the thickness of the body 110; (iii) transmission component to communicate sensor information to the body 110 and/or computer system 140.

In variations, the insertion mechanism 120 includes resources to enable data transmission and/or reception. For example, sensor information obtained through the sensor elements 144 can be communicated wirelessly or through other communication link to the mechanical body 110 and/or connected computing device. Likewise, in some implementations, information can be received on the insertion mechanism 120. For example, the insertion mechanism 120 can receive a feedback signal that is indicative of the motion or orientation of the insertion mechanism relative to a correct or incorrect use. As a more specific example, the insertion mechanism 120 can include, for example, a lighting element that illuminates to provide feedback as to the correctness of the use of the insertion mechanism 120. Still further, in some variations, the tip 122 can include a camera or optical component. Such an optical component can, for example, communicate a view for a virtual environment that is based on the position and movement of the tip of the insertion mechanism 120.

Sensor System

With reference to FIG. 1A and FIG. 1B, mechanical body 110 and/or insertion mechanism 120 can include a sensor system 140. The sensor system 140 provides a mechanism to detect and track the movement of the insertion member 120 as the insertion member 120 is inserted into the mechanical body 110. In one embodiment, the sensor system 140 detects the position of the tip 122 relative to the penetratable region 123, including whether the tip 122 pierces the penetratable region 123. As an addition or alternative, the sensor system 140 detects the position of the tip 122 relative to the tube 119 and/or the base of the shell 117. As another addition or variation, the sensor system 140 detects the angle of penetration of the tip 122 through the exterior layer 135. Still further, the sensor system 140 can detect the movement of the insertion mechanism 120, such as start and stops or fluidity of movement, as well as other information such as the linearity or efficiency of movement of the insertion mechanism 120.

In one embodiment, the sensor system 140 includes sensor elements that are provided with the mechanical body 110. The sensor system 140 can also include a sensor output mechanism 132 for transmitting sensor values and information to attached devices.

In some embodiments, the sensor system 140 includes sensors that are embedded or provided with the tissue layers 124 (including the exterior surface 135 or interior layers 137) and/or spinal model 116. Moreover, different types of sensors or sensing mechanisms can be employed within the mechanical body 110. For example, the sensor system 140 can include sensors for detecting orientation and/or position of the tip 122 relative to the mechanical body 110 (or elements of the mechanical body).

Examples of sensors that can be incorporated into the mechanical body 110 include deformation sensors, proximity or force sensors, orientation sensors, touch sensors or optical sensors. Still further, in implementation, the sensors can include magnetic sensors, capacitive sensors, resistive sensors, and/or optical sensors. Specific examples of sensors include Hall sensors and fiber optical sensors. Various sensing schematics can be used to detect, for example, angle of entry of the insertion mechanism 120 (e.g., proximity and/or optical sensors, orientation sensors on insertion mechanism), trajectory of the insertion mechanism 120 (sensors within mechanical body that detect deformation of tissue layer 124, or which come in contact as a result of the tissue layer 124 deforming), contact between the tip of the sensors and elements representing the spine or the spinal cord (e.g., tube 119) (e.g., touch sensors or proximity sensors). In one implementation, the sensing schematic is implemented so that the thickness of the mechanical body 110 is mapped to a coordinate system. When the tissue layer 124 deforms in select regions as a result of the insertion mechanism 124, the deformation 124 is mapped to the coordinate system, thus determining information such as trajectory, angle of entry, depth of insertion, etc.

As an addition or variation, sensors 111 can also be placed along elements representing “hard tissue”. Likewise, if fluid flows through the system physically then pressure and/or flow sensors can be placed along the tube. Otherwise, if virtual fluid runs through the cerebral spinal fluid space or vascular space, virtual pressure and flow would be displayed

As an alternative or variation, the insertion mechanism 120 can include components that comprise part of the sensor system 140 which transmits information relating to the positing of the insertion mechanism 120 relative to the mechanical body. For example, the insertion mechanism 120 can include the sensor element 144, which can include a sensor or sensor actuator. When implemented as a sensor, the sensor element 144 detects information about the orientation, position and movement of the insertion mechanism 120 within the mechanical body 110. When implemented as a sensor actuator, the sensor element 144 triggers or otherwise enables sensors of the mechanical body 110 determine information such as orientation, position, and relative movement. Thus, in one implementation, sensor element 144 to include metallic components which trigger output from sensors (e.g., Hall sensors) embedded with the mechanical body 110. This combination can provide another mechanism for the orientation and relative position of the tip 122 as the insertion mechanism is inserted into the mechanical body 110.

In some embodiments, the sensor system 140 can include a sensor output mechanism 132, which communicates sensor information from sensors of the insertion mechanism 120 and/or mechanical body 110. In one implementation, the sensor output mechanism 132 communicates sensor information, provided from, for example, sensors embedded in the mechanical body 110, to an output device such as a computing system. The sensor output mechanism 132 can, for example, include a communication port which connects to, for example, a properly equipped computer system or a wireless transceiver which communicates with such computer system. In a variation, the sensor output mechanism 132 can be provided with the insertion mechanism 120.

In some embodiments the sensor system 140 can include sensors in the mechanical body 110 which detect elements or portions of the insertion mechanism 120. Alternatively, the sensor system 140 includes elements that cooperate with corresponding elements of the insertion mechanism 120. For example, the sensor system 140 can include sensors that are distributed on the mechanical body 110 and on the insertion mechanism 120.

As described with FIG. 1C, an embodiment includes one or more sensor elements 144 provided on the insertion member 120 (e.g., on the tip 122). For example, the sensor(s) 144 of the insertion mechanism 120 can communicate with, or actuate sensors of the mechanical body 110. As another variation, the tip 122 of the insertion mechanism 120 can include a pressure transducer that detects pressure as the insertion mechanism 120 makes contact with the exterior layer 125, and/or is inserted into the interior layers 127. The mechanical body 110 can also include sensors or actuators for sensors, positioned to detect insertion of the insertion mechanism 120 through the penetrable region 123.

In another variation, the mechanical body 110 can include a distribution of sensors that are embedded near the exterior layer 135 and the interior layers 137. The sensor element 144 of the insertion mechanism 120 can be used in connection with sensors or microphones embedded within the mechanical body 110. Alternatively, the sensors can be placed on the needle and the transmitters can be provided within the tissue. To enhance the transmission of magnetic energy, metallic magnetic particles can be added to the mechanical body 110 to enhance transmission of the magnetic flux to the Hall sensors.

Still further, in an alternative variation, the sensor element 144 of the insertion mechanism 120 includes a magnet or sonic transmitter that is placed on its tip 122, and the mechanical body 110 includes a plurality of sensors 111 (e.g., Hall sensors, microphones) that are embedded within its synthetic tissue layers 114. In such an implementation, the sensor system of the mechanical body 110 detects the insertion, positioning and movement of the insertion mechanism 120. As an addition or alternative, metallic or magnetic particles can be added to the synthetic tissue layer 114 of the mechanical body 110. The addition of the magnetic or metallic elements can enhance the magnetic energy for sensor output.

Usage Implementation

Embodiment such as described with FIG. 1A through FIG. 1C can have a variety usage implementations. For example, mechanical system 100 can be implemented as a standalone system that enables a medical practitioner to practice the administration of a cerebrospinal needle or catheter injection. Alternatively, the mechanical body 110 can be utilized with conventional spinal needles or catheters or endoscopes. In either implementation, the user can receive tactile feedback that is indicative of whether the user correctly administered the simulated injection into the mechanical body 110.

As an alternative, the mechanical body 110 an/or the insertion mechanism 120 can record information regarding the insertion, movement and positioning of the insertion mechanism within the mechanical body 110. The recorded information can then be connected to an output source, such as a computer or display medium, where information about the user's operation of the mechanical system 100 can be evaluated and communicated to the user.

Still further, some embodiments provide for virtual environment that further enhances the simulation provided with mechanical system 100. FIG. 1D illustrates an embodiment in which the mechanical system 100 is used in connection with the virtual simulation 180 of a spinal region that is receiving a spinal injection. The virtual simulation 180 can be generated by computing system, using a display 182 or other display medium. An example of a computing system for generating the virtual simulation 180 is described with FIG. 2. The virtual simulation 180 displays a graphic model of a spinal region that is also physically represented by the mechanical body 110. The insertion of the insertion mechanism 120 can be replicated virtually on the graphic model in real-time.

With further reference to FIG. 1D, many aspects of the human body and response can be simulated for the user virtually. For example, aspects of the human body (e.g., aorta, fluid flows such as aortic flow, nerve roots, small vessels, abnormalities such as scar tissue) can be simulated virtually rather than physically.

In a variation, a camera window representing a needle's eye view can be included as part of the virtual environment. For example, the camera view can be provided as a window that is superimposed as a small screen within a screen (on screen display) along with the other traditional anatomic representations.

As another variation, the angle of insertion of the insertion mechanism 120, the movement of the insertion mechanism 120 within the synthetic tissue layers, and the relative position of the insertion mechanism 120 to the spinal model 116 can be detected by sensor system 140, then communicated to a computing environment and represented graphically in the virtual simulation 180. Other characteristics regarding the use of the insertion mechanism 120 can include the fluidity of its motion as it reaches its target (e.g., does the user stop and start), and the angle of the insertion mechanism after penetration.

In some embodiments, the virtual environment can also augment the realness of the mechanical simulation. For example, while the mechanical body 110 may comprise spinal model 116 and tissue layers 124, other aspects such as nerve roots, blood vessels, fluids, organs etc. can be represented virtually. Moreover, these aspects can be coordinated and made dependent on the physical events that occur with the model 110 with the insertion mechanism 120. For example, the sensor system can convey events such as tissue deformation, which in turn serve as input to affect virtualized aspects in the virtual environment 182. As described with some embodiments, a model for the spinal region can integrate virtualized aspects that respond to the events of the physical environment (e.g., insertion mechanism that misses the penetratable region 123 etc.).

Additionally, in some variations, when the insertion device traverses physical coordinates that represent virtualized or real anatomic hazards, the system can respond via feedback to alert the user. Such feedback may be auditory, tactile, visual, etc. The feedback may represent a patient movement or vocalization. Alternatively, the feedback may be computer implemented feedback that alerts the user of the error, or alternatively anticipates an error (e.g., based on snapshot data, such as the angle of entry) and provides an alert and/or feedback.

System for Virtual Implementation

FIG. 2 illustrates an example system for virtually representing a simulation of a spinal region procedure in connection with a physical model such as described with FIG. 1A through FIG. 1D. A system 200 can be implemented on a computing device, or combination of computing devices, to communicate with a mechanical system 250 for purpose of creating a virtual environment to enhance or augment the physical simulation provided by the mechanical system 250. The mechanical system 250 shown in FIG. 2 can correspond to, or be implemented using, for example, the mechanical body 110 and/or the insertion mechanism 120 shown by any of the embodiments of FIG. 1A through FIG. 1D.

In an embodiment, the system 200 includes a sensor interface 210, a real-time virtualization component 220, and virtual environment 230. The virtual environment 230 can correspond to a run-time environment for a computer program that models the spinal region of a class of subjects (e.g., adults, adult males or females, children, dogs etc.). Accordingly, the virtual environment 230 includes graphics, and optionally audio, to simulate the environment represented by the mechanical body 110. For example, the spinal region of a “patient” can be displayed to include animation, images (e.g., X-ray etc.) or video, displaying a spine, tissue, fluid movements, skin and other aspects of the human body. The virtual environment 230 can be generated with the execution of processes and algorithms that are based on a model of, for example, a human cerebrospinal and/or neurovascular or vascular system. In some variations, actuators can be structured to cause movement of the mannequin, such as to mechanically simulate a cough or pain induced movement.

Multiple human models can be maintained in a model library 232, and each model can provide algorithms, data and processes for recreating the virtual environment 235 for a particular kind of patient. For example, separate models can be maintained for adults versus children, men versus women, and/or human versus animal parentheses e.g., courses, dogs etc.). Moreover, the model library 232 can include models that account for anatomical hazards, such as injuries, abnormalities (e.g., cysts or tumors), or other known medical issues that can arise with humans in the context of the administration of several spinal punctures and injections based on years of accumulated malpractice data and practitioner experience which can be assembled into a database. Each model can include graphics (e.g., animation, images, and video) that form a virtual framework 235 for the virtual environment. When system 200 is in operation, the virtual environment 230 can use instructions and data (“model data 231”) from an appropriate model of the model library 232 to generate the virtual framework 235. In this way, the virtual framework 235 provides the baseline graphic and/or audio representation of the segment of the human body, based on a selected model.

In operation, sensor interface 200 receives sensor information 211 from the mechanical system 250. The mechanical system 250 includes the mechanical body 212 and the insertion mechanism 214. The mechanical system 250 can include a sensor output mechanism 202 provided with the mechanical body 212. The sensor output mechanism 202 can utilize sensor information 211 obtained from one or more sensors 213 distributed on or within the body 212) e.g., as described with FIG. 1B) and/or insertion mechanism 214 (e.g., as described with FIG. 1C). For example, with reference to FIG. 1B, the sensor output mechanism 132 of the mechanical body 110 can output sensor information to a coupled computer or computing device. The sensor output mechanism 202 can also be provided by the insertion mechanism 214. For example, with reference to FIG. 1C, the tip 122 of the insertion mechanism 120 can include the sensor element 144, which can correspond to a sensor, sensor actuator, or sensor output mechanism.

In one embodiment, the sensors 213 are distributed in the mechanical system 250 in accordance with one or more coordinate systems 201. For example, individual sensors can have their own coordinate system, and/or a collection of sensors (e.g., residing in the mechanical body 212) can be distributed based on a coordinate system. The physical reference frame 201 for the sensors 213 can, for example, capture deformation of tissue layers within the body 212, contact or proximity of the tip of the insertion mechanism to various points of interest etc. The coordinate systems 201 of the sensors can be integrated with a virtual coordinate system, as described below.

The real-time virtualization component 220 can process sensor information 211 received through the sensor interface 210. The virtualization component 220 can generate real-time virtual content (“RTVC 222”) representing the orientation and position of the insertion mechanism 214 relative to the mechanical body 212. For example, RTVC 222 can include virtual content representing events such as (i) the angle or orientation of the tent of the insertion mechanism 214 as it punctures the exterior layer of the mechanical body 212, (ii) the trajectory of the insertion mechanism 214 within the mechanical body 212, (iii) the fluidity in the movement of the insertion mechanism 214 (e.g., whether there is stop and start within the tissue layer of the mechanical body) and other motion parameters (e.g., velocity or acceleration of the insertion mechanism 214 relative to the mechanical body 212); and/or (iv) the relative position of the tip of the insertion mechanism 214 relative to aspects of the mechanical body 212 (e.g., relative to tube 119, shell 116, arterial tube etc.).

The real-time events provided by the insertion mechanism 214 and the mechanical body 212 can be rendered using RTVC 222 by the real-time virtualization component 220. The RTVC 222 can be overlaid or otherwise integrated with the virtual framework 235, so as to superimpose of integrate the events corresponding to the insertion of the insertion mechanism 214 within the mechanical body 212. The virtual framework 235 and real-time virtual content 222 can be combined into a real-time virtual output 238 that simulates the user's manipulation of the mechanical system 250 with a living body. In particular, the virtual environment 235 can maintain a virtual coordinate system. Events detected in the mechanical body 212 can be detected by sensors 213 and communicated based on the coordinate system utilized by the respective sensor(s). The RT virtualization component 220 and/or virtual environment 230 can map events detected from the sensors 213 into the virtual coordinate system 229, thus enabling spatial events of the mechanical body to be virtually represented. For example, sensors 213 can be distributed to deform when the tissue layers of the body 212 are deformed. These deformations can be captured through sensors 213 and communicated into the virtual output 238 by mapping the deformations from the reference frame 201 of the sensors into the virtual reference frame.

Additionally, in some embodiments, the model that provides the virtual framework 235 may virtualize some real-world elements, such as nerve roots, blood vessels or flows, organs etc. The model can include logic to respond to events, such as placement of the insertion mechanism, angle of entry, trajectory etc., and the response can be reflected in both the framework 235 and the virtualized aspects.

In this way, the virtual output 238 illustrates in real-time the effects of the user's manipulation of the mechanical system 250. The illustration of the user's manipulation of the mechanical system 250 can be made in the context of a virtual environment that represents, for example, the relevant portion of the human body. Among other benefits, such virtualization further augments and enhanced the experience of the user, by providing a more visual, contextual and responsive environment from which the user can learn and progress.

In some embodiments, an evaluation component 240 can run to evaluate the performance of a user who administers the puncture being simulated by the insertion mechanism 214 and the mechanical body 212. The evaluation component 240 can maintain evaluation parameters 242 that defines skill level, standards or criteria, or other milestones that relate to skill level. By way of example, the evaluation parameters 242 can correspond to one more of the following: (i) angle of entry of the insertion mechanism 214, (ii) depth of penetration, (iii) whether the penetrable region 123 (see FIG. 1) was penetrated, (iv) the relative position of the tip 122 of the insertion mechanism 214 relative to the spinal cord (e.g., tube 119) or spine (e.g., shell 117), and/or (v) the fluidity of the motion.

The evaluation component 240 can reference sensor information 211 reflecting the events of the user's simulation performance against the evaluation parameters in order to generate an evaluation output 244. The evaluation output 244 can reflect a programmatic evaluation of how the user performed in the simulation, based on sensor information 211 (or interpreted information) as well as evaluation parameters 242. The evaluation output 242 can correspond to, for example, a score, ranking, a grade and/or commentary, based on the detected evaluation parameters. In one embodiment, the evaluation component 240 includes criteria for determining whether an individual has a qualification or skill level to perform a procedure of the simulation in a real context.

In some variations, the perspective of the virtual framework can be changed. For example, it could reflect a side view that is omniscient, a practitioner view, or a needle eye view. Thus, the models used for the virtual projection can be changed in terms of the viewpoint that they depict while the physical model is being manipulated.

According to some embodiments, multiple representations can be displayed on the screen at once with a picture within a picture or onscreen display. Orientations can be changed with user input or can implemented with sensors on the practitioner or device to automatically change perspective. Deep anatomy can be displayed on computer eyewear or video projectors and superimposed on the physical anatomy to allow the user to develop mental imagery of the underlying anatomy.

Methodology

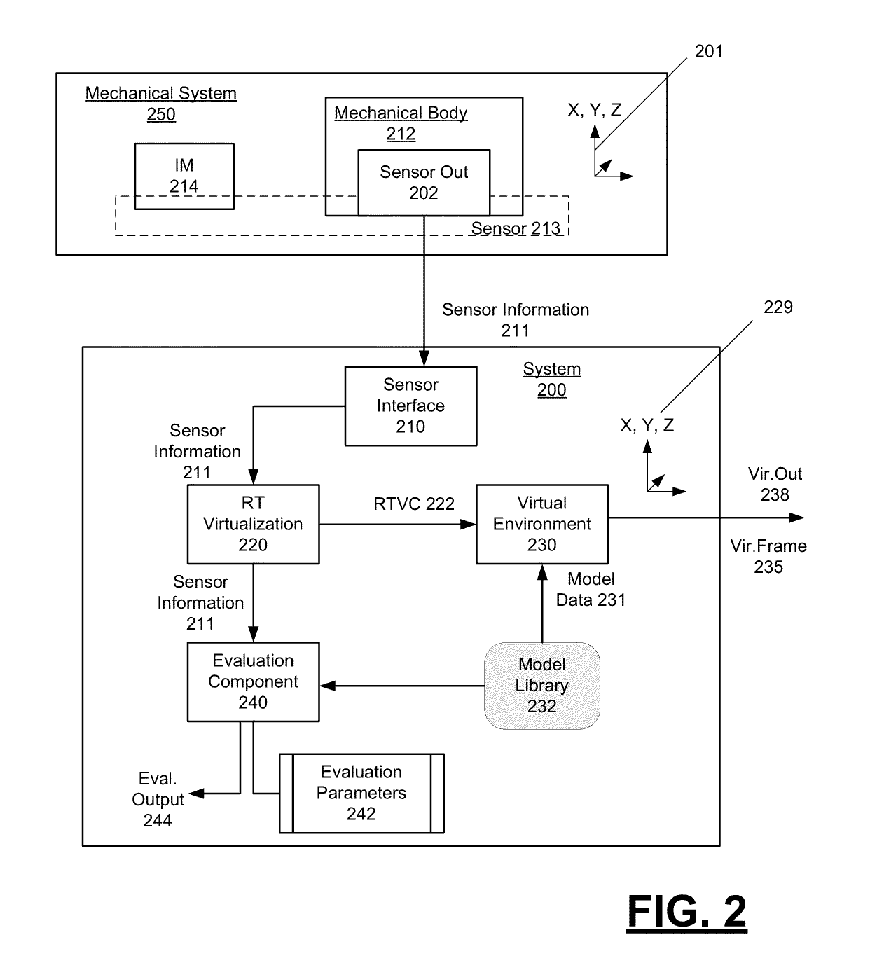

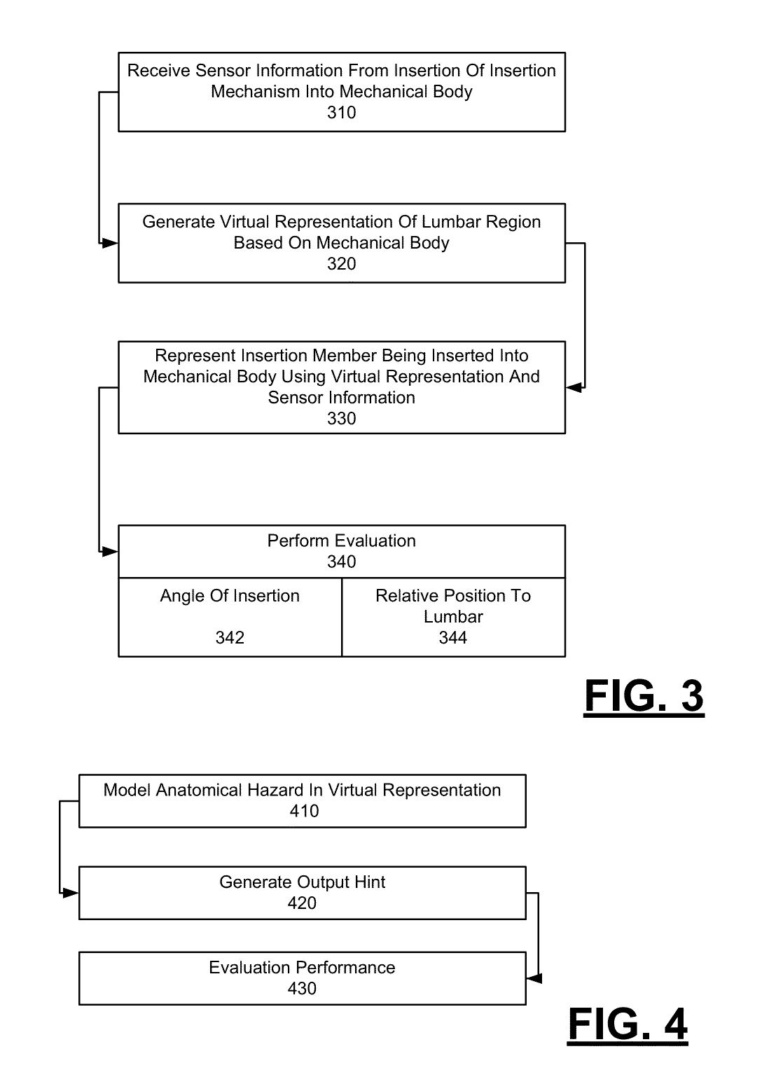

FIG. 3 illustrates a method for generating a virtual representation of a spinal region puncture based on a mechanical simulation, under an embodiment. FIG. 4 illustrates a method a method for simulating the presence of an anatomical hazard when generating a virtual representation of a spinal region puncture based on a mechanical simulation, under an embodiment. In describing example methods of FIG. 3 and FIG. 4, reference may be made to elements described with other embodiments, including with FIG. 1A through FIG. 1D and FIG. 2, for purpose of illustrating suitable components or elements for performing a step or sub-step being described.

With reference to FIG. 3, a computing device can generate a virtual representation of a spinal region (310). The virtual representation may be linked or associated with the mechanical body, so that events of the mechanical body are reflected in the virtual representation.

Sensor information is received corresponding to the insertion of insertion mechanism 120 into the mechanical body 110 (320). The sensor information can be received by a computing device that is connected to the mechanical system 100. For example, the sensor information can be communicated by the sensor output mechanism 132 of the mechanical body 110 to the computing device. Among other items of information, the sensor information can provide, for example, (i) angle of entry of the insertion mechanism 120 within the mechanical body 110; (ii) penetration depth of the insertion mechanism; (iii) a continuous tracking of the position (trajectory) of the tip 122 of the insertion mechanism 120 as it nears the spinal cord (e.g., the tube 119 of the mechanical body 110); (iv) the fluidity of the trajectory as the insertion mechanism 120 is inserted to the target location and withdrawn; and/or (v) the collision of the tip 122 with any unintended element of the mechanical body (signifying a mistake by the user). In implementation, the computing device that receives the sensor information can also generate a virtual representation of a spinal region that is being simulated through the mechanical system.

The manipulation of the insertion mechanism 120 within the mechanical body is simultaneously represented in the virtual environment (330). More specifically, sensor information can communicate events regarding the positioning, orientation and movement of the insertion mechanism 120 relative to the body 120. For example, the events can be graphically represented in the virtual environment. In some embodiments, the events conveyed through the sensor information are translated into virtual content that illustrates position and movement of the insertion mechanism 120 in relation to the body 110. The events can be conveyed in the virtual environment in real-time.

Additionally, some real-world elements that are dynamically affected by the penetration of the insertion member within the human body can be virtualized (e.g. blood, nerve roots). The virtualized aspects can be programmed with logic to be dependent, and affected, by the insertion member 120. The affect of virtualized elements can be modeled on physiologic responses, reflected in the selected model 132, so that the virtualized aspects are then made responsive to the insertion mechanism 120 in a manner that based on aspects such as position and trajectory of the insertion mechanism 120. Physiological responses can be determined with advanced computer models. Anatomic representations can be animated or predicted with complex algorithms such as finite element analysis or computational fluid dynamics.

In some embodiments, an evaluation can be performed relating to the manner in which the insertion mechanism 120 is used (340). The evaluation can factor in various aspects of how the insertion mechanism 120 can be used. For example, the evaluation can be based on sensor information that identifies an angle of insertion for the insertion mechanism (342). As another example, the evaluation can be based on sensor information that identifies a position of the insertion mechanism 120 (e.g., the tip 122) relative to other elements of the mechanical body 110 (344).

In addition to evaluation, some embodiments can provide for a virtual environment that provides real-time feedback to the user. The real-time feedback can be used as a mechanism to instruct or guide the user. For example, if the user's angle of entry is off, then real-time feedback can detect the error and signal a message or indication (e.g., light) to prompt the user to correct the position. Similar feedback can be provided to the user for other aspects of the process, such as the trajectory, velocity, fluid of motion, and depth/location of the insertion mechanism 120.

In some implementations, the logic employed can project, for example, an outcome of the simulated procedure based on a current course of action by the user as the user begins or advances the insertion mechanism 120 into or near the mechanical body 110. In such implementations, the feedback signaled to the user can be anticipatory or predictive, in relation to what is conveyed by the sensor information.

With reference to FIG. 4, an anatomical hazard can be modeled for the simulation environment (410). For example, the virtual representation of the spinal region can account for the patient (or virtual patient) to include scar tissue at a location that obstructs or is proximity to the insertion mechanism.

In one implementation, the computing system generates an output hint that is indicative of the anatomical hazard (420). The response of the user to the output can then be evaluated (430). By way of example, the user may change technique, angle of entry or perform other safety measures. As an example, the message “Ow, that hurts a lot” can be spoken from a computer that is coupled to the mechanical body 110. When the audible is heard by the user, then the user has the opportunity to consider the possibility of a hazard. If the user assumes the hazard is present, then the user's manipulation of the insertion mechanism 120 relative to the mechanical body 110 can account for the hazard that the user believes is present. The user can evaluated based on whether the user was correct to assume the presence of the anatomical hazard, as well as the manner in which the insertion mechanism 120 and the mechanical body 110 were used in relation to the anatomical hazard.

In some embodiments, the evaluation can be standardized. For example, a set of criteria can be predefined for purpose of defining the skill level of the user. The criteria can, for example, be used to certify a practitioner, so that a skill level of the practitioner can be judged without living subjects. The criteria can include, for example, metrics of insertion (e.g., angle of entry, angle of insertion, fluidity of movement, depth penetration, success rate, anatomical hazard detection/avoidance etc.). In some variations, the results of the practitioner can be compared to real world results, so as to enable prediction of the pracititioner's ability or skill level.

Computer System

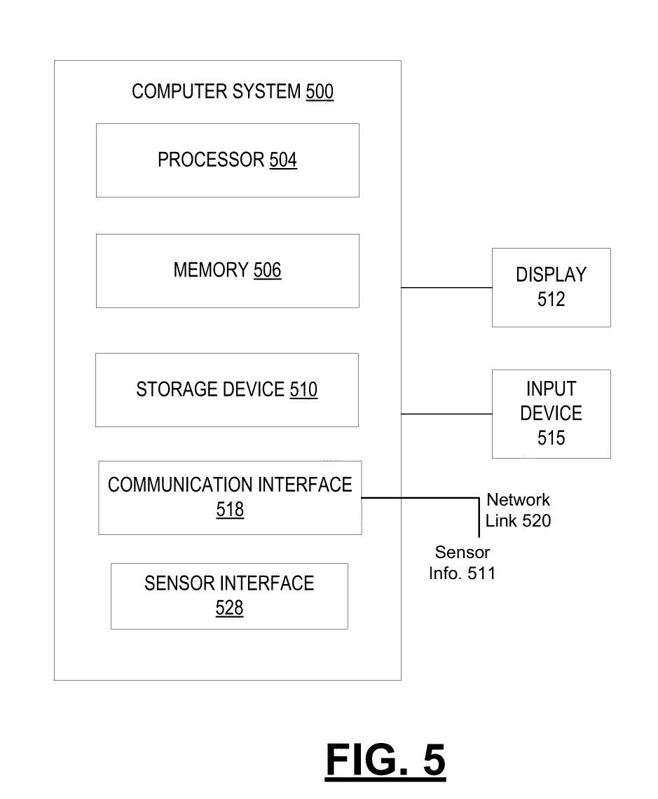

FIG. 5 is a block diagram that illustrates a computer system upon which embodiments described herein may be implemented. For example, in the context of FIG. 1, system 100 may be implemented using one or more computer systems such as described by FIG. 5.

In an embodiment, computer system 500 includes processor 504, memory 506 (including non-transitory memory), storage device 510, and communication interface 518. The memory 506 can include one or more of a main memory, such as a random access memory (RAM) or other dynamic storage device, for storing information and instructions to be executed by processor 504. Main memory 506 also may be used for storing temporary variables or other intermediate information during execution of instructions to be executed by processor 504. Computer system 500 may also include a read only memory (ROM) or other static storage device for storing static information and instructions for processor 504. A storage device 510, such as a magnetic disk or optical disk, is provided for storing information and instructions. The communication interface 518 may enable the computer system 500 to communicate with one or more networks through use of the network link 520 (wireless or wireline).

Computer system 500 can include display 512, such as an LCD monitor, and a television set, for displaying information to a user. The display 512 can be used to display, for example, the virtual output 238 (see FIG. 2). An input device 515, including alphanumeric and other keys, is coupled to computer system 500 for communicating information and command selections to processor 504. A sensor interface 528 (e.g., wireline or wireless link) can also be provided to receive sensor information 511 (e.g., from the communication link 518). According to some embodiments, the memory 506 can store instructions and data corresponding to one or more models of the spinal region. The processor 504 can generate a virtual environment of, for example, the spinal region in context of a simultaneous mechanical simulation. The memory 506 can also store instructions for processing sensor information 511 as events reflected in the virtual environment. Thus, components and logic described with elements of FIG. 2 can be implemented through the computing device 500. Furthermore, example methods such as described with FIG. 3 and FIG. 4 can be implemented using the computing system 500.

Some examples described herein further include computer implemented methods, such as described with FIG. 2 and FIG. 3. According to one embodiment, those techniques are performed by computer system 500 in response to processor 504 executing one or more sequences of one or more instructions contained in main memory 506. Such instructions may be read into main memory 506 from another machine-readable medium, such as storage device 510. Execution of the sequences of instructions contained in main memory 506 causes processor 504 to perform the process steps described herein. In alternative embodiments, hard-wired circuitry may be used in place of or in combination with software instructions to implement embodiments described herein. Thus, embodiments described are not limited to any specific combination of hardware circuitry and software.

In one implementation, a computer is connected to a mechanical body and a display screen. The computer can operate a system such as described with an example of FIG. 2, and display a virtual simulation such as shown in FIG. 1D. In variations, other kinds of computing devices can also be used. For example, a portable device (e.g., such as provided by cellular devices) can be used as a medium to display the virtual output 238 (see also FIG. 2). Alternatively, computing goggles or eyeware can be used to display virtual content to the user.

Although illustrative embodiments have been described in detail herein with reference to the accompanying drawings, variations to specific embodiments and details are encompassed by this disclosure. It is intended that the scope of embodiments described herein be defined by claims and their equivalents. Furthermore, it is contemplated that a particular feature described, either individually or as part of an embodiment, can be combined with other individually described features, or parts of other embodiments. Thus, absence of describing combinations should not preclude the inventor(s) from claiming rights to such combinations.

What is claimed is:

1. A system comprising:
a mechanical body comprising a spinal element, and a synthetic tissue layer;
one or more sensors that are provided with or coupled to the mechanical body;
one or more processors that communicate with the one or more sensors to detect insertion of an insertion device into the mechanical body, wherein the one or more processors operate to:
provide a virtual representation of a spinal region that corresponds to the mechanical body, the virtual representation representing the insertion device being inserted into the mechanical body based on a movement and/or position of the insertion mechanism in relation to the spinal element and the synthetic tissue layer of the mechanical body.

2. The system of claim 1, wherein the one or more processors provide the virtual representation by outputting on a display a representation of the insertion mechanism as the insertion mechanism is inserted into the mechanical body.

3. The system of claim 1, wherein the one or more processors output the display in real-time in response to movement of the insertion mechanism within the mechanical body.

4. The system of claim 1, wherein the one or more processors operate to:
provide, with the virtual representation, a representation of one or more fluid circuits, wherein the virtual representation of the one or more fluid circuits are affected by movement and/or position of the insertion mechanism in relation to the spinal element and the synthetic tissue layer of the mechanical body.

5. The system of claim 1, wherein the one or more fluid circuits represent at least an artery about the spinal region.

6. The system of claim 1, wherein the mechanical body further comprises one or more fluid circuits that mechanically simulate the spinal region.

7. The system of claim 1, further comprising the insertion device, wherein the insertion device communicates with at least one of the mechanical body or the one or more processors.

8. The system of claim 7, further comprising the insertion device, wherein the insertion device includes one or more of the sensors.

9. The system of claim 1, wherein the one or more processors use information provided from the one or more sensors to evaluate how a user inserts the insertion mechanism into the mechanical body.

10. The system of claim 9, wherein the one or more processors use information provided from the one or more sensors to evaluate an angle of insertion of the insertion mechanism.

11. The system of claim 10, wherein the one or more processors use information provided from the one or more sensors to evaluate a positioning of the insertion mechanism relative to the spinal element.

12. The system of claim 1, wherein the synthetic tissue layer includes one or more layers that are physically modeled to provide a tactile feel of at least one or more of skin, subcutaneous tissue, or ligament tissue.

13. The system of claim 1, wherein the one or more processors provide the virtual representation to include an anatomical hazard.

14. The system of claim 13, wherein the one or more processors communicate a message that is indicative of the anatomical hazard as the insertion mechanism is inserted into the mechanical body.

15. A computer-implemented method receiving sensor information that indicates insertion of an insertion mechanism into a mechanical body;
generating a virtual representation of a spinal region; and
representing graphically, as part of the virtual representation, the insertion mechanism being inserted into the mechanical body.

16. The method of claim 15, wherein representing the insertion mechanism is performed in real-time, in response to sensor information generated by the insertion mechanism being inserted into the mechanical body.

17. The method of claim 15, generating the virtual representation includes simulating one or more anatomical hazards, and providing output that is indicative of the anatomical hazard in response to the positioning or movement of the insertion mechanism within the mechanical body.

18. The method of claim 15, further comprising evaluating how the insertion mechanism is inserted into the mechanical body in connection with outputting the virtual representation to a user who is viewing the graphic representation of the insertion mechanism being inserted into the mechanical body.

19. The method of claim 17, wherein evaluating how the insertion mechanism is inserted into the mechanical body includes evaluating an angle of insertion of the insertion mechanism.

20. The method of claim 19, wherein evaluating how the insertion mechanism is inserted into the mechanical body includes evaluating a relative position of insertion of the insertion mechanism relative to the spinal region.

21. The method of claim 15, wherein generating the virtual representation includes simulating graphically the presence of tissue, bone and fluids of a human spinal region.

22. A mechanical body comprising:
a spinal element and one or more synthetic tissue layers;
a plurality of sensors structured to detect insertion of an insertion mechanism; and
a communication link to communicate an output of the plurality of sensors to a computing system in real-time.

23. The mechanical body of claim 22, wherein the plurality of sensors are structured to detect a position of the insertion mechanism relative to the spinal element.

24. The mechanical body of claim 22, wherein the plurality of sensors are structured to detect an angle of insertion of the insertion mechanism.

25. The mechanical body of claim 22, wherein the one or more synthetic tissue layers are structured to provide a tactile feedback that simulates a human tissue.

26. The mechanical body of claim 25, wherein the tactile feedback is variable with a thickness of the one or more synthetic tissue layers to simulate different kinds of synthetic tissue layers.
